SEQUENCE LISTING (11 sequences)

SEQ ID NO: 1 - DNA, 14128 bp, Parabacteroides goldsteinii
gtgttgaaaatcaaagattttattatcctcttctcgaagcaaaaatacttcaatcgctgggtaatcttttttatagacctgtttttctcggttttatccacttttttagtatttgcgacccttggcaatattttgaaaataagtctgccggtacgtgagtttatttatataaatctcttttccatcctatgcagtacaatttgttttctttcctgccagacctataaaggtgtcattcgtcattcaacttttacagagacggggcgtattgctattgcctcttttgtgaaagctactttgatgattgtagcccttttctgttttatccactcgttatcttttcttcaaatctttttaggaggtttgcttgatttattcctgactttctttattctaacaacgtttcgtgctttattaataatgatgtatagcatgatagttagaagtattgcttccaataatgagaagttattaatttatagggaagaaggtacatcatcaagctcatctgtaatttctctatggaaacaatccgatagttgtcagattggaggatatatacgaataggggagagaagtaaaattcgtataggaggctatcccgtttattctgtcaagaatcagatggaatttaatgctttagtggatcgaaaagggatcaagacgattttgtttaccgactatcatgtcgtaaaagcagaaagtgaacgtctggtgcgttattgcgagaaaaaaaagattcgtatgttggtattttcagcaatgaacgagttaaaaagcagaacagtcaacctgcataatttacctgagattcgtattgaagacctgttagggcgtgatgaaataaatatcaatctaaaaaaaatagcaggtgaattaaaaggaaaagttacgctggttacaggagccgccggttctatcggtagtgaactttgtcgtcaactatgtaaattaggattaaagcaattgatcttgtttgatagtgccgagacacctatgcatactcttcgtctggagttggaagatcgatatccggaagttgttttcactccgattatgggagatgtccggatgttggctcgagtggaaagtgtttttaaacgattccatcctcagtatgtttttcatgcggcagcctataaacatgttcccttgatggaagaaaacccttgcgaagctgtgcacaccaacgtccagggaacctgtaatatggcagacatgtctgtaaaatatgatgtagacaaatttatcatgatctctacggacaaggcggttaaccctacgaatgtgatgggagcctccaaacgtttggccgaaatctatgtacaaagtctgagtattgcgataaataaaggaatccatcctggtaaaactcgatttatcactactcgttttgggaatgtattagggagcaatggttctgttattcctcgttttcgtgaccaactggcaaaaggaggtcctttgaccgtcactcatcccgacatcatccgttattttatgacaattccggaggcctgtcgtcttgttttggaagctgcatttatgggagaagggaacgagatttttgtgtttgatatggggactccggtaaagatagccgatctggcacgtcgaatgatagagttggtaggtcttattcccgataaagacattgaaataaaatataccggtcttcgtccgggtgagaaattgtatgaagagttgttggcaactaaggaaaatacgcttcctaccataaatgctaaaattttccgtgcgaaagtgcgtgaatatgattttgccgagatatcagctggtattgatcatttagtagatttggcaatccgagtagaaaagttggaaaccgtaaaaatgatgaaatcgatcgtgccggagtttataagtcggaattcggagtatgagaaactggataagtaggaagaagtaatatatagcgattgttggtttaggtttgaataagaaagaatatttttaatattctctcaaataggaagatggaaaatgttaataaatcaaatatcaatatgtcgaaagaaattaagattgccgtagctggaaccggttacgtaggattgagtattgccaccttgctggcgcaacatcatgcagtgatggctgtggatgtcattcctgagaaggtggaaatgttaaaccgcaagcagtcgcctattcaggatgagtatattgagaaatatttggttgagaaagagttggatttgacggccacgcttgatggggcttccgcttatcgtgatgctgatttcattgttatagcggctccaacaaactacgatccagcgaagaattatttcgacacgcaccacattgaggatgtaatcgatttggtattaagtgtcaatcctgaagccgtaatggtgatcaagtctacgattcccgtgggttattgtcgtagcctctatttgaggtacgctcaaaaagggatacggaagttgaatttactgttttctccggagttcctgcgtgagagtaaagccctttacgataatctatacccgagtcgtatcatcgtaggtttcccgaaactgattacagatgaacggttcaaggaagagaatgaggccattcgtcgcatcgctgatattccagctttagaaaaagcagctcatattttcgccgacttgttgcgggaaggtgctatcaagcaagatatacccacgcttttcatgggtatgaaggaggccgaggccgttaagttgttcgccaatacctatctcgctttacgtgttagttatttcaatgagttggatacctatgcggaagtgaagggattggaatcgcaagctatcattcagggagtagggttggatccacgcattggtactcattataataacccgagttttggttatggtggttactgtcttcccaaggataccaagcagttgttggccaattatcaagacattcctcaaaatatgatgaccgccattgtggagagtaaccggacacgtaaggacttcattgcggatcaggtattacgtaaggccggttattatagttatagtgatcggaacagtttctgtcatgcagaagagaaggattgcgtgataggtatttatcgcctgacaatgaagtcgaacagcgacaatttccgtcaatcttctatccaaggcgtgatgaaacgcatcaaggcgaaaggagcatgtgtgatcgtttatgagcctacgctagaggatggagtttctttctttggtagtactgtggtgaacgaccttgttaagtttaaggagatgagccacgcaattattgccaaccgctatgacggttgtctggatgatgtaatggggaaagtatatacgagggatatttttaatagggattaggattcatatccaaagatgatatttttgaaggaaaaactgggtaggaataagactgtattggcgaatttctcctacctgagcatattgcaggtatttacgatcctgttccccttgttgacttatccttatttgttgcgtgtcatagggttggaattatatggggtgattgtatttgcccaaacgattgtcaactatgtctcgttggtaataaatttcggatttaatatgtccggggcaagaaatgtcgctgtttataaggacgacaagacccgattatcaaatattgtatcatctacttatttatgtaaattgatcttatgggttatttgtcttgtcgtatatttatcagttataagcatagtcccgttttttgaggatcattattgggtctacgcactctcttttctgttgacttttaatgagcttctgttacctatctggttcttccaagggattgagaagatgaagtatatcacgatcgtgaatttgtcagcccgtctgttattcgtggttgctatcttcttgtttgtccatgagcgggaggattatctgatagttcctttattgaatggtattggtgctatgttggcgggttgcttgtcattgtatatcgtattgggaaaggagaggataaggctgtccgtgatacctatacgaaaattaaaatcggcttatagggaaagttttccattatttatttccaacttgtctacccagatttatgtgaatgtgaacaaattggtaataggttcttttttgggaatgtcggaagtctctatctatgacatggctgataaggtgttgcatttgatgaagctccctatttccatgatggcgcaagccgttttcccgaagatttcccgagaacgtaatatccttttcgtgaatcgtgtgatgtttttggtggtgggaattgtgctgttggcttatatctgtgtgtttatcggtagtgaccagatcgtgtattttttcaccgggaagtatatgagagaagcttctattatcatgcgtttattggggatatccgctatcttggtttcatttaatacttttataggaggaaatcggcttgtccctatggggtattcgtctgtctatatgaaggtgatggttggtaattgtttgttttttttggtagggataggcttgcttttgcttacacataatataaatatgtatacggtgacagtgatggtggtgacagtagaaggttttagctttgtttctttagtttatagaaattggcgattaggattgttgagaataaaaagataaagaaagtggcatcgggagattagtgtttaaagaatagatttcgtgtcttttttgagatagtattggttgcatcgtttttgatgtgaggagtggataggtcaaataaatttttgtttattctcttgtagttttggaagttttagtgtctcaattttttatgtaagagaatgaaaaagtaaaaataagagaaatagcattgaaagtggtttcaaaacataaaagatgattatgttttatgggaaatataaataggagtatagtgctatcctttagggatgtattgttatggtttactttttttatagtaatagggagtgcatgctttcaaactcaatacaaatttccattattattcgcattgttcaacatattgtcgaaagtaatttttgtgacaagtttaatccttcacatcataagatgtttgagatataagaaacagttttttacgaagtatgatattttattgttgttactgttttcctggttagtgtgttcttctatactaaatggtcatagttggattatgcaggttccttttgtaatgaaatctctttcgattcgttatatctttacattgtatactcctaatcgcaaagaaaagataataaccattttgtcatctgtttttacatttattgtttatttgaatttttttcaattactctttttttcatcaataatgggttacgttgatggagataaagtctatcttatttcaacgaattataaccagtttggggcagtatttatgccggcgatttttttaaaaataatagaatcgtttcagacaggttgtaaaagaagtttgattttgttgtttgtagtctgtgtcgcttctgtcgcaatagaaggttcagcaacggcaacgatttctatattactattattacttactgttgtttttgttaaaaaatatttttttctaaaggtagaaaacatatcaatcttggttgggattgtattgttttttttcacatttgtcttaccagtactttcgatattcaatgtaccttttgttagtagttttgtagaaggtttgggaaaggatatgacattttcgcatagaacgtccatttggatttatgcgttatttcaaatatcttatagtccgctaataggttatggggtatgcgataaggagtggtttcagtttttgttgagtaatgcggtaaatactcataatgtcattttacattttttaatacaagggggaggggttggtttgttgatatttgcggtatttgtgtatatcggaattaaacaacttctgtctattcaagataagtaccaaagagagttgttgctgacagtagtaattgttttcttcttaatgtctcagtttgaagtatatactaatacattcatatatatatttatatttttaatgtttatatccaaagaaatgcttacttgtaaattcattgaaaatgtttaattatactcttataataccgcattacagatctgttgatacattacatagattgcttttatcaattcctgataggaatgatatccagatattggtgattgatgataatagtagtttggatgaagacgaatttaaacgtttgccgttatttgcttttgctgaatttttaataatttttttaatgaaaaacgaaggggccggaggcgctagaaataaggggctaaagacagccaaagggaagtggctgatatttgcagattctgatgattattttgttgaaaatgcattttctatatttgataaatataataatgaagattatgacatagtttattttaaatcggagagtatctgtttgggcggtgacattgtttctgataggagtaattattataatcaattgattgatagtttttcacctgaagatgtgaagtctgtaaacaagttgagatacaatcatggtgtaccatggtgcaaaatgataaaaaaaaatttggtaatattcaataatatccaatttgatgctataatgtatagtaatgatgtaatgttttctacgaaaataggatactatgcagaaaaaatagcggctgtcaatagtattgtttattacgttactacgaacgtaggtagtttgacatcccaaaaaagtaaagaatcattgttatgtagatatgaaacagctcttagacaaaataaatttttaagagatagagaattggctaagtatcagaaatctattatgttttatcttagattatcactgaattatggtgtttcaacattgctgttttttattagattaggaataaaatatcgtgcaaattttacaataggaatgcttcattggcatgaatatataactactaaaattaagtctaagtgtttgttttagttgtatctcgaggttttccttcttctttggatgtgatgaatgggaattttgaggcggatcaagttagggcactacgagctttaggtcataaggttgtcgtatttagcatagacagacgttttaacgcttgtgatagacatattggtcttaatcatagatttgtagatggaatagatgtgtataatttctatttgttaccaatgccgataaagactatgtataaattaggttatttttatatagttcagatggctaaattaatgtattggatcattcttcgaaatcatggtaagccagacatcatacatgcacattatctgtatactatgcccattgctttaaatttgaagagcagattcaaaataccgtgtgtcggtactgagcattggagctttgtaggaaaaagtaatataccgaatcatgttaaattttatgctgaaaatgtgtatcctaaattggatgctcttattactgtttcaaatagtttaaaaacaaatataagtaaacaatttggtgtggagagcattgttataccgaatatgttggatatatctaaatttaatatcgaagctcaatgtgattctaataaagacggagcgtatttttcttttatatcggttggtgcgcttgttggaggaaaatgctttgatttattgcttaaagcattctcaaagttgaaatttgaaaataagtgtttaacaatcattggggatggacctcaaaagcaagatttgaaaaacttgattatagaattaggccttcaagattatgtagagcttacaggtaaattgactagagatgaaatgttaccaaagttatcagtctcgaatatttttgtcttgccatcaaattctgaaacatttggggtcgtttatattgaagcaatggctttaggattacctgttattgctaccaagtgtggtggtcctgaagattttgtgaatttttctaatggtttgttgatagagcggaataacgaggaacagttgattgatgcaatggaatatatgtacagtcattataatgaatacaagccggaaataataagtaaggatattataaataaatactcgccaagtaaagttgctaggcagatagaatctttatataatgagtgtttatgctactaagaggtattggtcgagcatcttagcaaatcattgtttgtcaattagaaatccttaatattgattatcactacatttgcttatgagttgtctgaattttttataaaataatgctaaacatgatgtaatataatgagggaaaatatgaaaggttatttatttgtcagcaattcttctaaaccggggcatattatcagtgaacctaaaactgttaatgttgattcttttgctttgcctgcaatttatgcggctgataagctgggatataaattgtatataggaatcaactcaaataatccaaagttgataacttgtaagcaatatgatgtcgtttattacaaccaaaatagttttcgaaatccatttgctattaaagataacataaaagcatataagaatctttgtgcctttttggagaaaaattcaaatatagaagtaattcattgtaatactcctataggtggtgtgattggtagactttgtggttgtaaataccacaaaaaagtgatatatactgctcatggatttcactttttcaaaggggcttcaatagtgaattggttgctttattatcctatagagaaatggttggctcattataccgatgctctcatcacgattaataaagaggactacgagagagctaaaaaattcaagcttcgtaaaaatggtaaagtttattatgtaccaggtgtaggtgttgacttatctgtatttaacaaaggggaggataaatctgaattgaggaaagaatataatattaaagatgatgaaatagtgtttgtctcaatgggtgacttagttcctcgtaagaactatgctgttgcattgaacgctatagctaaagctggagtaaataatctcaaatatcttatctgtggaaacggtcctcaaatggatgaaatgaaacaactctgcgtaaaacttggtattgaaaaacaagttgaatttcttggtagaagaggcgatgtatcgaagatcgtgaaggcctctgatgtatttctattcacgtcaaaacaagaaggcttagctcgttctctgatggaagcaatggcaagtggcttaccttgtgttgtttctgctattcgtggtaacacagatttgattgataatgaaaaaggaggatttatgtatgatgtaacagatattgatggttttgtggaaggtatcaagaggatttgtggagatagtgaacttcgccaaaaaatgggaaactataatttattaaaaataaagaactttgatattaatgcttcaaaagctgcattttatactgtgtttaaacaagaattaataaaaggtatataagctatgtataagtgtttttttaaaagactgatagacatagtaggatcgttgtgtatctgtccttttgtggtgttggaaattgtaatattagcacctctaatttatttcacggatagaggtccgattttttttaatggtcagagagtaggtaaaggtggtaagatttttaaaatgtttaagttacgttcaatgtacatcaattctaaagacatacgtaatgctgatggttcgacatttaattcagacaatgatccacgtgttactcctattggtaggtttattcgtaagaccagtttagatgaatttcctcaattcttcaatgtcctaataggtgatatgagtctggtaggtccacgtccatctttgacaacaactccgtactcagagtatagtgaagtaagaaagaagagagtatcgatctgtcctggtgtgacaggttactctcaggcatactaccgtaactcgataggtcaagatgagaagttcaagtatgatgcttattatgcagataacgtaacattttggatggatattaaagttttatttaagacattttggactgttattgcaaacaagaatattaatacgggcgaatcaatgaagaaataatgaaaaaaaaaatacttataattggagcttcaatactccagcttccagcaattaaaaaggcaaaagagatggggttagaggtagctgtagccgactttaatccagaggcaataggagtaccatatgctgacaaatatttcaatgcaagtactattgacattgaagcaattgtaagagttgctgaggaatatcaaccggatgggataataacattggcaacagatatgccaatgagaagcatagctgttgccacaagcaagctaggtcttgtgggaattacttctgatacagctttgaaatcaactgataaggctgaaatgattaaggcttttcgctctgcaggtgtaccggttccgtggtattatattgttgataataaggctgtttttaatgaaaattttgctgaatttactttcccttgtatcatgaaacctacggataattcaggaagccgtggtgttgtcttgattaagtcggttgacgaattggttgatgcttataaatatagtcgtgagcagtctagaaatggtagtgttattgttgaggaatatttgcaaggtcatgaagtgagtgttgaagtgattgtctataagcaaggttcacatactgcaagtgactgacaagttaactacaggagctccaaattttgtggagatggggcattcacaaccatctcgctttattggtgaagccttggataaaattcacgatgttgcaacaaaagcatgtgctgctgttggtattgagaatggtccagttcatgttgagatgatggtaacagagtgtggaccgaagatgatcgagctaggagcaagaatggggggggactgtatcgctacgcatcttgttccattgtcaacaggtattgatatgattaaagcaacgattgacatagctctagggaacaaacctgacctggaacggaaattgtattgtggtagtgctattagatatttcgatttgccactaggtacaattaaatctataacaggagaagaagatgctctgaatgaggaaggtgtgtgtgatatcatgtttactaaacatgttggagataaggtgacttcaatacatagtagtcttgacagagtagggatgatagtctgtcaggcaaagtcagcggaagcagctgtcgcacaatgtataaaagcaaaagagaaagtgatgtttgaaatagaataatatgatagctagcaagaaactattgttcttaggtgggggacgtagcaatataaatgcaataaagtcagccttggaactcggtataaagccatacgttgtaggtatggaaggtaattatccaggttataaattggcagagaagattttcatagctaatattatggataagtacgatgttatgaaagctattaaagatgaaaagattgatggcgttttaatttgctgttcggatagagctatagagactgttggtttcttaaacgatcatttaggattgaatggtatcacagaatctgctgcaaaacgttgtaacaacaagtatgaaatgaagcaggctctttttaataagggggttaatactgcgaagttcatgaagcttgaaaaggaggcagacttgaagagggcgttagagttactgacattccctgtcatagtgaaagctgttgatcttcagagtagcaaaggtgtctacatatgtaaggatgaaacagaattgatggagaattactgtaaatcaatcagtgagtctaatcttacatactgcattgtagaggaatttatagaaggtagggaacttggtgctcaagcttttgtttgtaatggtaaaattgtatttgtgcttcctcacggtgatataattgctcatgagggtaagtcgaccgttcctgttggccattatgcaccattagattgtagtgaaaaactgcgcaagagaatcgccgaagaagcaggtaaagctatcgatgcacttgagttgaataattgtgcggttaatatcgattttattgttaaagatgatgttccatacattttagaactgagtggaagagttggtgcaaattgtctcccggaaatgacagggtatcactatggaatggattattataagatgattgttgctgtagctgttggtgaagatccattttcatatttcacccctaatatgaagggtaagataactttagttaagatgatcaattcagcagaaacgggtgtacttgaattacttgaatatgataaggacatgctgccttatgttagcttctttgtaaataagggggataaagtttcttcttttaaaaactccaatgactgtatcggtgaaatgttagttcagggcgacacaatggaagaatgtgaagaaaagataagaaaattctatgagaaacttgtgataaggttcaaatgatattatgcaaagaataagcaagacgagtataagcctttgtgcttcgggtattgcgcaacaagattttgagaatagcaggtgtaaatttatatcctaaatagaatattaaaattatttcttatgaagaaagttcctttttctcctcctgatataactgagagtgaggttaatctagtatcagaggctcttcgttccggttggatcactacgggacctaagacaaaagaatttgagagattaattgccatgtgctgtcagacggagcaagctgtatgcttgaattcggctacggcttgtatggagttgatattgcgtgttttgggtattggaccaggagacgaggtgattacttccgcttatacgtatactgctaccgcaagcgtaacttgccatgtgggggcaaaggttgtcatggtagataccgctcctaattcgtttgagatggattatgataagttggcagatgcgattacggagaggacaaaggttgttttgcctgttgatcttgcgggtgtggtttgtgattatgataaaatattcgctgtcgtagagagcaagaaacatttgttttcgccagcgaacgatatccagaaggcatacggtcgggttatcgtacttgcggatgccgctcatgcttttggtgccaaatggcatggaaagatgtgtggtgagatcgctgattttacttcgttttccttccatgcagtgaaaaaccttacgacagcagagggaggtgcgttgacttggagaaatcatgatggggttgacaacgagtctctttataaacagtttcaattgctttcccttcatgggcagaacaaggacgcacttgctaaaacgaggttgggggcatgggagtatgatattgtcgctccatattataagtgtaatatgacggatgtgatggcgggtattggtctagcgcagttgaagcgttatccggaaatgttgtatcgtcgtcgtcagattattgagaggtataatgaaggactgaaggggtgcgatgtgcaggtcttggatcattttggtgatgaccattcttcgagtgggcatttgtatcttgtacgtttgcttggtgaggatgtggaatatcgaaatgctgtgatagagcggatggctgaacgtgggatcgcttgtaacgtacattataaacctttaccgatgatgacggcctataaaaatttaggttttgatatcgtagattatccgaatgcttacaaccaataccataacgagattactcttcctttgcataccagtcttacggatgaggacgtagaatatgtgatctctaattttgttgatattattacccaataaaataatcaatcgatttgaaacgtttttttgatgttgtagccagataagaagggacttattatggtaggagggcgtgatccacgggttacacggtctggctattatatccgtaagtataaattggatgagtttcctcaattgatcaatgtactcaaaggggatatgagtttggtcggtccccggcccgaggtgcgtaagtatgtcgacatgtacacggctgaacaacggcgtgtgtgggcagtcaggccgggtattacagattatgcatctatagtgtatatggatgaaaataggttactgggccagtcctcaaatcccgacaaaacttatgttgaggatattatgccggcgaaaattgaactcaatatgagatatattaatcatcctacattgtttgagtactttaagttgatagttcttacgatttttaaaatagttcgataa

SEQ ID NO: 2 - DNA, 20 bp, artificial sequence
  PCR primer
attgccatgtgctgtcagac

SEQ ID NO: 3 - DNA, 20 bp, artificial sequence
  PCR primer
tcaccacacatctttccatg

SEQ ID NO: 4 - DNA, 21 bp, artificial sequence
  PCR primer
gagtataagcctttgtgcttc

SEQ ID NO: 5 - DNA, 20 bp, artificial sequence
  PCR primer
tcgttgtcaaccccatcatg

SEQ ID NO: 6 - DNA, 20 bp, artificial sequence
  PCR primer
caacaagccagggatgtaac

SEQ ID NO: 7 - DNA, 20 bp, artificial sequence
  PCR primer
gtttcgaacaatttgtatcg

SEQ ID NO: 8 - DNA, 20 bp, artificial sequence
  PCR primer
cacttataatatggagcgac

SEQ ID NO: 9 - DNA, 20 bp, artificial sequence
  PCR primer
aaagttcctttttctcctcc

SEQ ID NO: 10 - DNA, 21 bp, artificial sequence
  PCR primer
catctttgaaataggtgcagg

SEQ ID NO: 11 - DNA, 21 bp, artificial sequence
  PCR primer
cctctgccattaacagcaatg

(19) United States
(12) Patent Application Publication
(10) Pub. No.: US 2021/0115478 A1
(43) Pub. Date: Apr. 22, 2021

(54) METHOD OF PRODUCING SORBITOL USING PARABACTEROIDES GOLDSTEINII

(71) Applicant: Multistars Biotechnology Company Limited, Taoyuan City (TW)

(72) Inventors: Tzu-Lung Lin, Taoyuan City (TW); Po-I Wu, Taoyuan City (TW)

(21) Appl. No.: 16/747,582

(22) Filed: Jan. 21, 2020

(30) Foreign Application Priority Data
Oct. 17, 2019 (TW) 108137536

(51) Int. Cl. C12P 7/18

(52) U.S. Cl. CPC C12P 7/18

(57) ABSTRACT

The present invention provides a method for sorbitol production comprising: providing a Parabacteroides goldsteinii strain, cultivating the Parabacteroides goldsteinii strain in a suitable cultivation medium, and contacting the Parabacteroides goldsteinii strain with the hydrophobic substrate to form sorbitol to replace the disadvantages produced in industrial manufacturing. Because it is possible to control the production of sorbitol according to the demand by regulating the expression level of genes related to the production of sorbitol in the Parabacteroides goldsteinii, it enables sorbitol to be more effectively applied to products of a food, a medicine, or a skin care product.

### CROSS-REFERENCE TO RELATED APPLICATION

This application claims priority of Taiwan patent application No. 108137536, filed on Oct. 17, 2019 the content of which are incorporated herein in its entirety by reference.

### BACKGROUND OF THE INVENTION

This application incorporates by reference the material in the sequence listing submitted via ASCII text file titled Method_of_producing_sorbito_using_parabacteroides_goldsteinii_Sequence_listing, with the date of creation being Sep. 16, 2019, and the size of the ASCII text file in bytes being 20,504.

### 2. The Prior Art

Many bacteria contain glycocalyx on their surfaces of cells. The main function of glycocalyx is to help bacteria survive from harsh environments and resist to immune system attacks from hosts. Glycocalyx with compact structure on the surface of bacteria would form capsule, while with loose structure on the surface of bacteria would form slime layer. According to previous studies, the reason that the bacteria in the gastrointestinal flora can exist in the gastrointestinal tract of the host for a long time and stably is mainly related to the specific capsule polysaccharides on the surface of these kids of bacteria.

Parabacteroides goldsteinii is a multi-functional probiotic bacterium. Many studies have pointed out that the polysaccharides on the surface of the bacteria are related to attach to the surface of hosts or adapt to the physiological environment of hosts. Furthermore, Parabacteroides goldsteinii is a probiotic strain that has only recently been isolated and studied, and no research has been conducted to explore the entire genome, coding genes, or metabolism of Parabacteroides goldsteinii. Therefore, in order to better understand the adaptability of Parabacteroides goldsteinii in the gastrointestinal tract of an individual, it is necessary to further analyze the genes related to the polysaccharide synthesis of the bacterial capsule in the Parabacteroides goldsteinii of the present invention, and then further explore the efficacy of these genes or its roles in the bacteria.

Sorbitol is a hexahydric alcohol that is widely existed in various fruits in nature, such as apples, peaches, dates, plums, and pears. Sorbitol is the main raw material for the synthesis of vitamin C or sorbose. Sorbitol has a refreshing sweetness but the sweetness of it is only about 60% of sucrose, and it only contains 2.6-3.3 calories per gram, which is lower than the 4 calories provided per gram from other carbohydrates. Therefore, sorbitol is often used in weight-loss or low-calorie foods; furthermore, sorbitol is not regulated by insulin in human metabolism, so the blood glucose level rises slowly after consumption. Sorbitol is mainly metabolized in the liver through the action of enzymes to produce fructose, and is then used by the body in the form of fructose. Therefore, sorbitol is often used as a substitute for sucrose in foods for patients with diabetes. Moreover, sorbitol is not used by harmful bacteria in the mouth, so it is often added to chewing gum to prevent dental caries, and it has a cool sweet taste, so it can also be used as a sweetener in sugar-free chewing gum; in addition, sorbitol has the function of moisturizing and preserving, and it is one of the earliest sugar alcohols allowed as a food additive, so it can be used to improve the moisturizing property of foods or as a thickener. Therefore, sorbitol is often used in baked foods to extend the shelf life thereof, and sorbitol is often used in the toothpaste industry to replace glycerin as a moisturizer and excipient. In addition, sorbitol can also be used in cosmetics as a humectant and excipient.

However, currently available sorbitol is industrial manufactured and is not a product from natural synthesis; wherein, the main method for industrial production of sorbitol is to reduce glucose; for example, the glucose solution is heated, pressurized, and catalytically hydrogenated under the catalyst of nickel to produce the raw materials of sorbitol, which is then decoloring and removing heavy metal ions to obtain pure sorbitol.

Although industrial manufacturing can obtain a large amount of sorbitol, after obtaining the raw materials of sorbitol, it still needs to go through many purification steps, which not only results in the production of industrial waste, but also could be affected by differences in the quality control of the purification process. The quality of sorbitol products which is not uniform or poor would makes it troublesome when applied to other products.

In summary, it is necessary to develop a method of naturally producing sorbitol; and if probiotics can be used to produce natural sorbitol to replace industrially manufactured sorbitol, in addition to reducing the aforementioned disadvantages, it can also combine the effects produced by the probiotics themselves, so that sorbitol can be more effectively used in low-calorie or diet foods, foods for diabetics, prevention of dental caries, and moisturization; wherein, if the genes that regulate sorbitol production in this probiotic strain is found, the production of the sorbitol could be controlled by regulating these genes.

### SUMMARY OF THE INVENTION

An objective of the present invention is to provide a method for sorbitol production comprising: (a) providing a Parabacteroides goldsteinii strain; (b) cultivating the Parabacteroides goldsteinii strain in a suitable cultivation medium; and (c) contacting the Parabacteroides goldsteinii strain with the hydrophobic substrate to form sorbitol.

The other objective of the present invention is to provide a sorbitol-containing composition comprises a Parabacteroides goldsteinii strain.

In one embodiment of the present invention, the sorbitol is produced by a gene of capsular polysaccharide (CPS) of the Parabacteroides goldsteinii strain; wherein, the gene of capsular polysaccharide is located in a polysaccharide A gene region of the Parabacteroides goldsteinii strain, and the Parabacteroides goldsteinii strain is Parabacteroides goldsteinii DSM32939.

The Parabacteroides goldsteinii of the present invention is a multi-functional novel probiotic bacterium. In order to more fully understand the adaptability of the probiotic bacterium to the gastrointestinal tract of individuals, the present invention further analyzes and predicts the genes in the Parabacteroides goldsteinii that are involved in the synthesis of polysaccharides of bacterial capsules and further explore the efficacy or the role of the genes in the bacteria. After the predictive analysis, in the whole genome of the Parabacteroides goldsteinii of the present invention, it is found that the nine-segment CPS regions which may be a sequence fragment of the capsular polysaccharide gene region; wherein, because CPS region A (CPSA, i.e. gene region A, SEQ ID NO. 1) carries a nucleic acid sequence which is similar with the wcfR gene and wcfS gene in the polysaccharide A (PSA) gene region of the Bacteroides fragilis strain NCTC9343, which are mainly responsible for the synthesis of the capsular polysaccharide.

Therefore, this gene region in the Parabacteroides goldsteinii DSM32939 is defined as a capsular polysaccharide synthesis gene region, and subsequent studies are carried out. The plasmid containing the gene region A is prepared by conjugation, and the homologous gene recombination method is used to embed the plasmid in the wcfR gene to destroy the coding structure. By polymerase chain reaction, it is confirmed that the plasmid sequence has been successfully embedded in the wcfR gene of the Parabacteroides goldsteinii and caused the destruction of DNA structure. The reverse transcription reaction and polymerase chain reaction have also confirmed that when the DNA structure of the wcfR gene on the Parabacteroides goldsteinii is destroyed by inserting a plasmid in it, the RNA synthesis would indeed be destroyed.

Moreover, a gas chromatograph-time of flight mass spectrometer is used to analyze and compare the differences between the liquid culture metabolites of the native strain of the Parabacteroides goldsteinii DSM32939 and the mutant strain of the Parabacteroides goldsteinii DSM32939-wcfR′ with knock out in capsular polysaccharide gene. It is found that the content of sorbitol in the metabolites of the native strain of the Parabacteroides goldsteinii DSM32939 is significantly higher than that of the mutant strain of the Parabacteroides goldsteinii DSM32939-wcfR′, indicating that the Parabacteroides goldsteinii DSM32939 of the present invention can produce sorbitol, and the sorbitol production is regulated by genes involved in regulating capsular polysaccharides.

The present invention utilizes the Parabacteroides goldsteinii to produce natural sorbitol to replace the disadvantages caused by industrial manufacturing. At the same time, because the Parabacteroides goldsteinii itself is a multi-functional probiotic bacterium, it can also be combined with its own benefits as probiotics with the function of producing sorbitol to more effectively be used in low-calorie or diet foods, foods for diabetics, preventing dental caries, increasing moisturization, etc. In addition, the present invention can control related sorbitol production genes through regulating the Parabacteroides goldsteinii, and can control the production of sorbitol according to the demand; therefore, the Parabacteroides goldsteinii can be used to prepare sorbitol, and can be used to make a sorbitol-containing composition comprising the a Parabacteroides goldsteinii; wherein, the composition is a food, a drink, a nutritional supplement, a care product, or a medicine, and the composition is in a form of a powder, a granule, a solution, or a gel can be administered to a subject in need by oral administration or the like.

The embodiments of the present invention are further described with the following drawings. The following embodiments are given to illustrate the present invention and are not intended to limit the scope of the present invention, and those having ordinary skill in the art can make some modifications and refinements without departing from the spirit and scope of the present invention. Therefore, the scope of the present invention is defined by the scope of the appended claims.

### BRIEF DESCRIPTION OF THE DRAWINGS

FIG. 1 shows the schematic diagram of the predicted region of the capsular polysaccharide genes of the Parabacteroides goldsteinii.

FIG. 2A shows that the construction of the mutant strain of the capsular polysaccharide synthesis gene region of the Parabacteroides goldsteinii.

FIG. 2B shows the electrophoresis image that confirms the destruction on DNA structure of the capsular polysaccharide synthesis gene region of the Parabacteroides goldsteinii.

FIG. 3A shows schematic diagram of the test method for confirming the RNA expression of the Parabacteroides goldsteinii with the mutation on capsular polysaccharide synthesis gene region.

FIG. 3B shows the electrophoresis image that confirms the destruction on RNA expression of the Parabacteroides goldsteinii with the mutation on capsular polysaccharide synthesis gene region.

FIG. 3C shows the electrophoresis image that confirms the destruction on RNA expression of the Parabacteroides goldsteinii with the mutation on capsular polysaccharide synthesis gene region.

FIG. 4 shows the line chart of blank sample detection by gas chromatography-time of flight mass spectrometer to examine the situation of substance residues during the detection process of metabolites from the Parabacteroides goldsteinii.

FIG. 5A shows the mass spectrum of substance analysis in metabolites of the native strain and the mutant strain of the Parabacteroides goldsteinii.

FIG. 5B shows the bar graph of the expression level of sorbitol detected in the native strain and the mutant strain of the Parabacteroides goldsteinii.

### DETAILED DESCRIPTION OF THE PREFERRED EMBODIMENT

The data provides in the present invention represent approximated, experimental values that vary within a range of ±20%, preferably ±10%, and most preferably ±5%.

Statistical analysis is performed using Excel software. Data are expressed as mean ± standard deviation (SD), and differences between groups are statistically analyzed by one-way ANOVA.

### Definition

According to the present invention, the operating procedures and parameter conditions for bacterial culture are within the professional literacy and routine techniques of those having ordinary skill in the art.

The “metabolite” describes herein is a substance which is secreted into the bacterial culture solution after being metabolized by the bacteria, comprising the culture medium for culturing the bacteria.

According to the present invention, the pharmaceutical product could further comprise a pharmaceutically acceptable carrier that is widely used in pharmaceutical manufacturing techniques. For example, the pharmaceutically acceptable carrier can comprise one or more agents selected from the group consisting of a solvent, a buffer, an emulsifier, a suspending agent, a decomposer, a disintegrating agent, a dispersing agent, a binding agent, an excipient, a stabilizing agent, a chelating agent, a diluent, a gelling agent, a preservative, a wetting agent, a lubricant, an absorption delaying agent, a liposome, and the like. The selection and quantity of these reagents falls within the professional literacy and routine skills of those having ordinary skill in the art.

According to the present invention, the skin care product can be manufactured into a form suitable for skincare or makeup using techniques well known to those having ordinary skill in the art, including, but not limited to, an aqueous solution, an aqueous-alcohol solution or an oily solution, an oil-in-water type, a water-in-oil type or a composite type emulsion, gel, ointment, cream, mask, patch, pack, wipe, powder, aerosol, spray, lotion, slurry, paste, foam, dispersion, drop, mousse, sunblock, tonic water, foundation, makeup remover product, soap, and other body cleansing product.

According to the present invention, the food product can be used as a food additive, added by the conventional method in the preparation of the raw material, or added during the production of the food, and matched with any edible material to be made into food products for human and non-human animals

According to the present invention, the types of the food products include, but are not limited to, beverages, fermented foods, bakery products, health foods, and dietary supplements.

The Strain of the Parabacteroides goldsteinii of the Present Invention

Parabacteroides goldsteinii (hereinafter referred to as P. goldsteinii) MTS01 used in the examples of the present invention is a novel probiotic bacterium, which is deposited in Deutsche Sammlung von Mikroorganismen and Zellkulturen (DSMZ; Inhoffenstr. 7B, D-38124 Braunschweig, Germany) on Oct. 29, 2018, and the number is DSM 32939. P. goldsteinii is an obligate anaerobe that needs to be cultured in an anaerobic incubator at 37° C. for about 48 hours, wherein in the the examples of the present invention, the is cultured at 37° C. with Whitley DG250 Anaerobic Incubator (Don Whitley, UK), which is with an anaerobic environment mixed anaerobic gas (including CO2: 10%, N2: 80%, and H2: 10%), and an anaerobic indicator (Oxoid, UK) is used to confirm whether the environment reaches the anaerobic condition. Besides, the liquid culture medium of the P. goldsteinii is NIH thioglycollate broth (TGC II) (purchased from BD, USA, No. 225710), and the solid culture medium is Anaerobic blood agar plate (Ana. BAP) (purchased from CREATIVE LIFESCIENCES, Taiwan). The P. goldsteinii is stored in a −80° C. refrigerator for a long-term preservation, and the protective liquid is 25% glycerin. It does not need special cooling treatment and can be stored by freeze drying to stabilize its activity.

The present invention provides a use of Parabacteroides goldsteinii to produce sorbitol. The regulation of sorbitol production by the related genes involved in the regulation of capsular polysaccharides in the Parabacteroides goldsteinii of the present invention.

Meanwhile, the present invention also provides a sorbitol-containing composition comprising a Parabacteroides goldsteinii and a pharmaceutically acceptable carrier, wherein the sorbitol is produced by the Parabacteroides goldsteinii and the composition is a food, a drink, a nutritional supplement, a skin care product, or a pharmaceutical product.

The Parabacteroides goldsteinii of the present invention is a multi-effect probiotic, in order to more fully understand the adaptability of the probiotic to the gastrointestinal tract of individuals, and many studies have pointed out the polysaccharide on the surface of the bacteria (i.e. the capsule of the bacterium) is related to its attachment to the surface of the host or to its adaption to the physiological environment of the host, for example, resisting the immune response of hosts. In addition, because the genus Parabacteroides goldsteinii is a probiotic strain that has been isolated and studied recently, the study has not yet showed the whole genome, coding gene, or metabolite of Parabacteroides goldsteinii; therefore, the present invention is in order to further understand the adaptability of Parabacteroides goldsteinii to the gastrointestinal tract of individuals. The following examples further analyze and predict the genes involved in the synthesis of polysaccharides of bacterial capsules in Parabacteroides goldsteinii of the present invention, and further explore the efficacy or the role in the bacteria of the genes.

### EXAMPLE 1

Prediction of the Capsular Polysaccharide Synthesis Gene Region of the Parabacteroides goldsteinii

In the embodiment of the present invention was to predict the capsular polysaccharide synthesis gene region of the Parabacteroides goldsteinii MTS01 of the present invention; wherein the polysaccharide synthesis gene region of the bacterial capsule is generally composed of a plurality of genes, including glycosyltransferase, flippase, polysaccharide export protein, and polysaccharide polymerase, and most of them are in the same direction; therefore, in this embodiment, the principle to find the gene region of the capsular polysaccharide synthesis in the whole genome of the Parabacteroides goldsteinii MTS01 was followed.

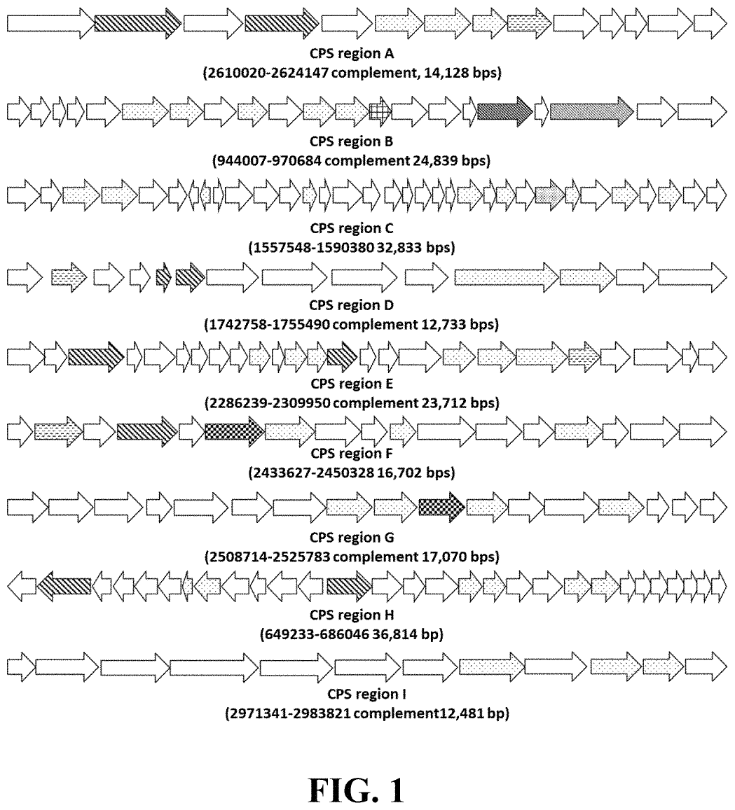

Each gene to be predicted was analyzed by a database that is available to the public, including NCBI, KEGG, and COG. The results of the analysis were shown in FIG. 1 that, in the whole genome of the Parabacteroides goldsteinii a MTS01 of the present invention, it was found that the nine-segment CPS regions which may be a sequence fragment of the capsular polysaccharide gene region, wherein each gene region was about 12-36 kb in size and each arrow in each gene region represented an open reading frame. Wherein, the glycosyltransferase was shown as a light gray dotted arrow, the sugar transferase was shown as a dark gray dotted arrow, the polysaccharide biosynthesis protein was shown as a slash arrow, the lipopolysaccharides (LPS) biosynthesis protein was shown as a checkered arrow, the capsule polysaccharide transporter was shown as light gray arrow, the capsule assembly protein was a shown as dark gray arrow, the capsule exopolysaccharide family protein was a shown as dotted square arrow, and the O-antigen ligase domain containing protein was shown as a checkerboard arrow, and other functional genes were shown as a white arrow.

CPS region A in FIG. 1 carries a nucleic acid sequence which is similar with the wcfR gene and wcfS gene in the polysaccharide A (PSA) gene region of the Bacteroides fragilis strain NCTC9343, and the protein sequence identity between the nucleic acid sequence in the CPS region A and the wcfR gene and wcfS gene in the Bacteroides fragilis were about 38.6% and 69.3%, respectively. Researches have shown that the wcfR gene and the wcfS gene of Bacteroides fragilis are mainly responsible for the synthesis of the capsular polysaccharide; therefore, in the present invention, this gene region in the Parabacteroides goldsteinii MTS01 was defined as a capsular polysaccharide synthesis gene region, and subsequent studies were carried out.

### EXAMPLE 2

Construction and Confirmation of Mutant Strain of the Parabacteroides goldsteinii in Capsular Polysaccharide Synthetic Gene Region

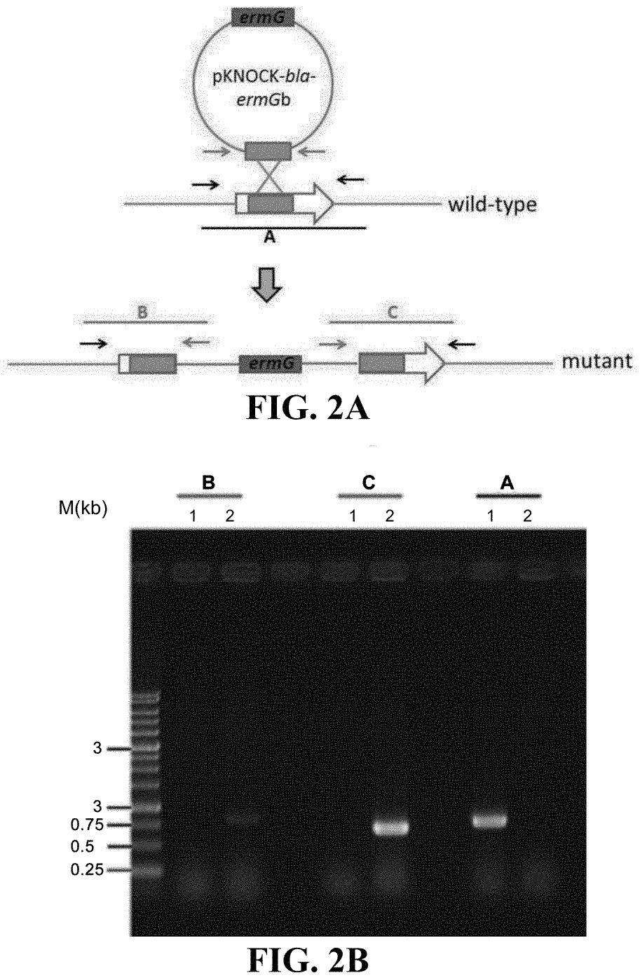

In the embodiment of the present invention was to construction and confirmation of a mutant strain of the Parabacteroides goldsteinii in capsular polysaccharide synthetic gene region (hereinafter referred to as MTS01-wcfR′) to confirm that the gene region A predicted in Example 1 was the capsular polysaccharide synthesis gene region of the bacterium. Considering that the wcfR (Aminosugar synthetase) gene is an important gene for the synthesis of capsular polysaccharide, it was therefore preferred in the present invention to destroy this gene to produce a capsular polysaccharide knock out in the Parabacteroides goldsteinii MTS01 of the present invention. The method for constructing the mutant strain MTS01-wcfR′ was shown in FIG. 2A; first, the primer of SEQ ID NO. 2 (5′-ATTGCCATGTGCTGTCAGAC-3′) and the primer of SEQ ID NO. 3 (5′-TCACCACACATCTTTCCAT G-3′) shown in Table 1 below was used to perform a polymerase chain reaction (PCR) to amplify the wcfR gene (see the gray square of A in FIG. 2A, wherein A represents the gene region A) of the Parabacteroides goldsteinii MTS01 and the amplified wcfR gene fragments contained a cleavage site of EcoRV at both ends. The amplified wcfR gene fragment was inserted into the pKNOCK-bla-ermGb plasmid with the EcoRV cleavage site using EcoRV, and the plasmid carries the ermG gene, which is a specific drug resistance gene and can be used to screen whether the bacterium is successfully carried in the plasmid.

TABLE 1
Primers for polymerase chain reaction

| Sequence number | Length of primers (ntds) | Length of products (ntds) |
|---|---|---|
| SEQ ID NO: 2 | 20 | 431 |
| SEQ ID NO: 3 | 20 | |

Next, the Parabacteroides goldsteinii MTS01 of the present invention was simultaneously mixed with Escherichia coli (E. coli) S17-1 λ-pir containing the constructed pKNOCK-bla-ermGb plasmid, and the two bacterium were placed on the filter paper for 36 hours in an aerobic environment for conjugation to transfer the pKNOCK-bla-ermGb plasmid constructed from the E. coli to the Parabacteroides goldsteinii MTS01 of the present invention. Then, as showing in FIG. 2A, after the plasmid transfer into the strain of the Parabacteroides goldsteinii MTS01 of the present invention, it corresponds to a fragment of the wcfR gene (i.e. the gray square region) on the genome of the Parabacteroides goldsteinii MTS01, and homologous recombination occurs to insert the plasmid into the gene of the native genome of the Parabacteroides goldsteinii MTS01 and destroy the wcfR gene, thereby obtaining the mutant strain of the Parabacteroides goldsteinii MTS01-wcfR' with mutation in the capsular polysaccharide synthetic gene. Next, the sheep blood agaric culture plate containing 4 μg/mL chloramphenicol and 10 μg/mL erythromycin was used to screen the mutant strains of MTS01-wcfR′. Finally, the pair of primers A, B, and C shown in Table 2 were used to confirm whether the mutant strain MTS01-wcfR′ was completed.

TABLE 2
Primer pair for polymerase chain reaction

| Sequence | Sequence | Length of primers | Length of products |
|---|---|---|---|
| pair | number | (ntds) | (ntds) |
| A | SEQ ID NO: 4 | 21 | 749 |
| | SEQ ID NO: 5 | 20 | |
| B | SEQ ID NO: 4 | 21 | 805 |
| | SEQ ID NO: 6 | 20 | |
| C | SEQ ID NO: 5 | 20 | 673 |
| | SEQ ID NO: 7 | 20 | |

Next, in order to confirm whether the capsular polysaccharide gene of the Parabacteroides goldsteinii MTS01 of the present invention has been successfully destroyed, the selected mutant strain of the Parabacteroides goldsteinii MTS01-wcfR′ (Mutant, M) with a capsular polysaccharide mutation and the native strain of the Parabacteroides goldsteinii MTS01 (Wild-type, W) were performed a polymerase chain reaction with primer pair A, B and C, respectively; and then, the size of the product obtained by the polymerase chain reaction was confirmed by agarose gel electrophoresis; wherein, if the wcfR gene of the Parabacteroides goldsteinii was successfully disrupted (i.e. the pKNOCK-bla-ermGb plasmid was successfully embedded), the mutant strain would lose the nucleic acid product which was visible at about 750 bp in the native strain. That is, performing the polymerase chain reaction with primer pair A, the nucleic acid product which was visible at about 750 bp could only be seen in the native strain, but not in the Parabacteroides goldsteinii MTS01-wcfR′; while, performing the polymerase chain reaction with primer pair B or C, the nucleic acid product which was visible at about 750 bp could only be seen in the mutant strain, but not in the native Parabacteroides goldsteinii MTS01.

The results of this experiment were shown in FIG. 2B. As shown in FIG. 2, performing the polymerase chain reaction with primer pair A, there was a nucleic acid product which was visible at about 750 bp only could be seen in the native strain; and no matter performing the polymerase chain reaction with primer pair B or C, there was a nucleic acid product which was visible at about 750 bp only could be seen in the mutant strain of the Parabacteroides goldsteinii MTS01-wcfR′, but not in the native strain. The results indicate that the method of homologous gene recombination in the embodiment of the present invention has successfully embedded a plasmid sequence in the wcfR gene of the Parabacteroides goldsteinii MTS01 of the present invention, and caused the destruction of the DNA structure of the gene.

### EXAMPLE 3

Impact of Disrupting the wcfR Gene of the Parabacteroides goldsteinii on RNA Synthesis

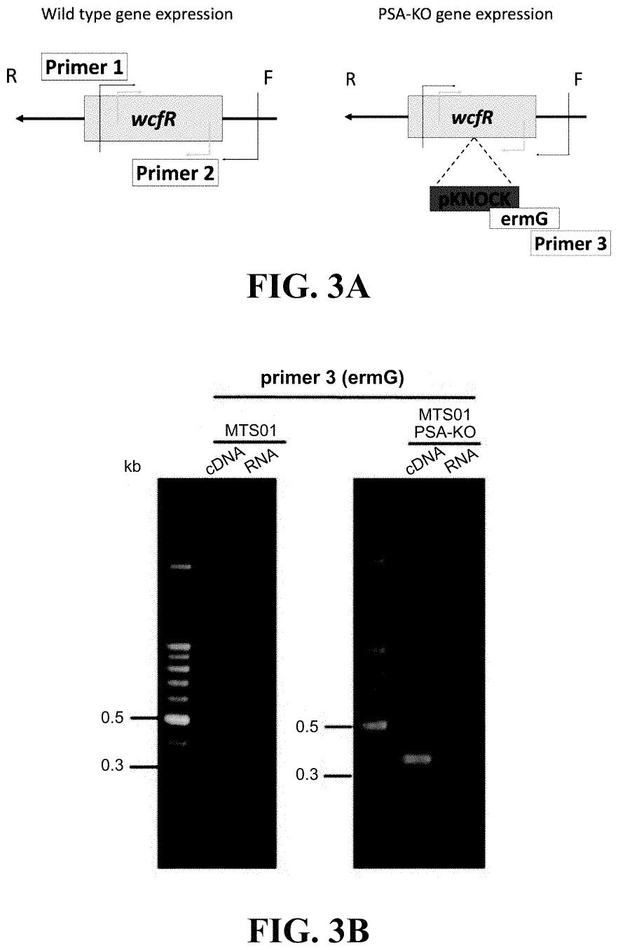

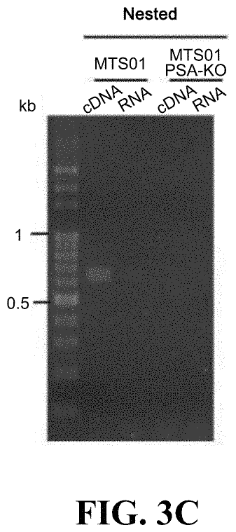

It has been confirmed in Example 2 that a plasmid was inserted into the wcfR gene of the Parabacteroides goldsteinii of the present invention to destroy its DNA structure, and the embodiment of the present invention would further determine the subsequent effects on the synthesis of RNA after the destruction of the DNA structure. As shown in FIG. 3A, in the example, three primer pairs were designed based on the wcfR gene, respectively Primer1, Primer2, and Primer3. First, RNeasy®MiniKit (purchased from Qiagen, Valencia, Calif., USA) was used to extract the total RNAs from the aforementioned the mutant strain of the Parabacteroides goldsteinii MTS01-wcfR′ and the native strain of the Parabacteroides goldsteinii MTS01 respectively. Then, Quant II rapid reverse transcriptase reagent kit (purchased from Tools, Taiwan) was used to perform reverse transcription reaction with the Primer1, Primer2, and Primer3 respectively and the extracted total RNA was as the template to obtain complementary DNA (cDNA), and then the polymerase chain reaction was used to compare the difference between the mutant strain of the Parabacteroides goldsteinii MTS01-wcfR′ and native strain MTS01, and RNA was used as the negative control group. If there were not PCR products with significant signal, the nested PCR, which is the special PCR that using the product from the first round polymerase chain reaction as the template for the second round polymerase chain reaction cycle, could be performed to improve the specificity and sensitivity of the product signal.

TABLE 3
Primer pair for polymerase chain reaction

| Sequence | Sequence | Length of primers | Length of products |
|---|---|---|---|
| pair | number | (ntds) | (ntds) |
| Primer1 | SEQ ID NO: 4 | 21 | 863 |
| | SEQ ID NO: 8 | 20 | |
| Primer2 | SEQ ID NO: 5 | 20 | 647 |
| | SEQ ID NO: 9 | 20 | |
| Primer3 | SEQ ID NO: 10 | 21 | 350 |
| | SEQ ID NO: 11 | 21 | |

Primer1 and Primer2 were both designed for amplifying the native wcfR gene; wherein, Primer1 was pg-wcfR-out R′-R plus pg-wcfR-out-F, and the resulting product should be 863 bp; while Primer2 was PSA-wcfR-out F-F plus pg-wcfR-R, the resulting product should be 647 bp; therefore, reverse transcription reaction and polymerase chain reaction with Primer1 and Primer2 would only amplify the native wcfR gene fragment, but not the disrupted wcfR gene. In addition, Primer3 was designed for amplifying the drug resistance gene on the plasmid of pKNOCK-bla-ermGb (PSA-KO); wherein, Primer3 was ermG-F plus ermG-R, the resulting product should be 350 bp; therefore, reverse transcription reaction and polymerase chain reaction with Primer3, only the strain with mutation of capsular polysaccharide would get the amplified fragment product only if the wcfR gene has been disrupted.

The experimental results were shown in FIGS. 3B and 3C. As shown in FIG. 3B, the reverse transcription reaction and the polymerase chain reaction with Primer3 could successfully obtain a cDNA product which was visible at about 350 bp in the mutant strain of the Parabacteroides goldsteinii MTS01-wcfR′ with knock out in capsular polysaccharide gene, and there was not any products with the same signal in the native strain. As shown in FIG. 3C, the reverse transcription reaction and nested polymerase chain reaction with Primer 2 could successfully obtain a cDNA product which was visible at about 647 bp in the native strain of the Parabacteroides goldsteinii MTS01, and there was not any products with the same signal in the mutant strain of the Parabacteroides goldsteinii MTS01-wcfR′. The results indicate that when the DNA structure of the wcfR gene on the Parabacteroides goldsteinii is destroyed by inserting a plasmid in it, the RNA synthesis would indeed be destroyed.

### EXAMPLE 4

Metabolites Analysis of the Parabacteroides goldsteinii

In the embodiment of the present invention, in order to confirm the real roles of the sequence predicted to be a capsular polysaccharide synthesis gene region in the Parabacteroides goldsteinii MTS01 of the present invention, and Gas chromatography-time-of-flight mass spectrometry (GC-TOF-MS) analysis was used to compare the differences between the liquid culture metabolites of the native strain of the Parabacteroides goldsteinii MTS01 and the mutant strain of the Parabacteroides goldsteinii MTS01-wcfR' with knock out in capsular polysaccharide gene, and the blank liquid culture medium was used as the control group.

First, the metabolites in each strain were extracted. 100 μL of the sample (i.e. the culture medium of the native strain MTS01 and the mutant strain MTS01-wcfR′) was placed in a 1.5 mL eppendorf, and 0.35 mL of methanol was added as an extract solvent. Then, 10 μL of adonitol was added as an internal standard, and evenly mixed on a shaker for 30 seconds, and then sonicated in an ice-water bath for 10 minutes. Next, the samples were centrifuged at 4° C. and 12000 rpm for 15 minutes, and then 0.34 mL of the supernatant of each sample were taken out into a new 1.5 mL eppendorf, and the extract was dried in a vacuum concentrator. After dried, 60 μL of the purified metabolite was added to the methoxyamine salt reagent (dissolved in 20 mg/mL pyridine), and gently mixed, and then reacted in an oven at 80° C. for 30 minutes. Next, 80 μL of N,O-Bis(trimethylsilyl) trifluoroacetamide (BSTFA) was added into each sample, which contained 1% of trimethylsilyl chloride (TMCS, v/v), and the mixtures were reacted at 70° C. for 1.5 hours. Then, Gas chromatography-time of flight mass spectrometry was used to analyze the instrument.

The Agilent 7890 Gas chromatography-time of flight mass spectrometer used in the embodiment was equipped with an Agilent DB-5MS capillary column (30 m×250 μm×0.25 μm, J&W Scientific, Folsom, Calif., USA). The specific analytical instrument parameters of the instrument were shown in Table 4.

TABLE 4

| Items | Parameters |
|---|---|
| Sample Volume | 1 μL |
| Front Inlet Mode | Split Mode |
| Front Inlet Septum Purge Flow | 3 mL min⁻¹ |
| Carrier Gas | Helium |
| Column | DB-5MS (30 m × 250 μm × 0.25 μm) |
| Column Flow | 1 mL min ⁻¹ |
| Oven Temperature Ramp | 50° C. hold on 0.5 min, raised to 320° |
| | C. at a rate of 20° C. min − 1, hold on |
| | 6 min |
| Front Injection Temperature | 280° C. |
| Oven Injection Temperature | 320° C. |
| on Source Temperature | 230° C. |
| Electron Energy | −70 eV |
| Mass Range | m/z: 75-650 |
| Acquisition Rate | 10 spectra per second |
| Solvent Delay | 3.833 min |

After detected by gas chromatography-time of flight mass spectrometry, MS-DIAL software was used to process and analyze peak data, baseline correction, deconvolution, peak integration, and peak alignment of raw data from all samples; wherein, a database of FiehnBinbase, which included matching of mass spectra and matching of retention time index, was used in the material qualitative work.

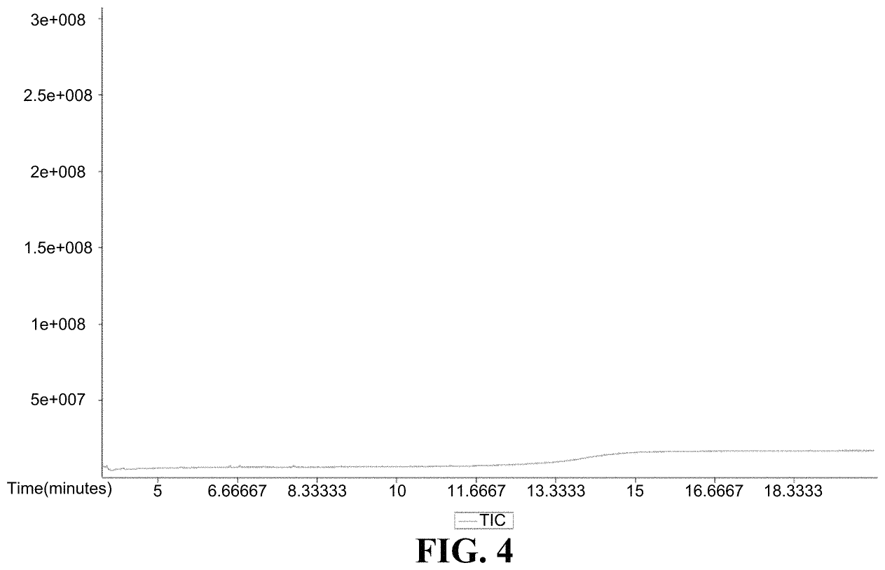

In the part of quality control, the blank sample was mainly used to check the residue of the substance during the test. The test results were shown in FIG. 4. As shown in FIG. 4, there was no significant peak detected in the blank sample, which indicated that example residual substances in the medium were well controlled and there was no cross-contamination between samples.

The known standards listed in Table 5 were used as internal standards, and the residence time of these standards was measured to confirm the experimental data and serve as a reference value for standardized experimental data.

TABLE 5

| FAMEs | RT (min) | Fiehn RI |
|---|---|---|
| C8 | 5.4870 | 262320 |
| C9 | 6.2362 | 323120 |
| C10 | 6.9540 | 381020 |
| C12 | 8.2770 | 487220 |
| C14 | 9.4630 | 582620 |
| C16 | 10.5370 | 668720 |
| C18 | 11.5130 | 747420 |
| C20 | 12.4070 | 819620 |
| C22 | 13.2330 | 886620 |
| C24 | 13.9960 | 948820 |
| C26 | 14.7760 | 1006900 |
| C28 | 15.7180 | 1061700 |
| C30 | 16.8480 | 1113100 |

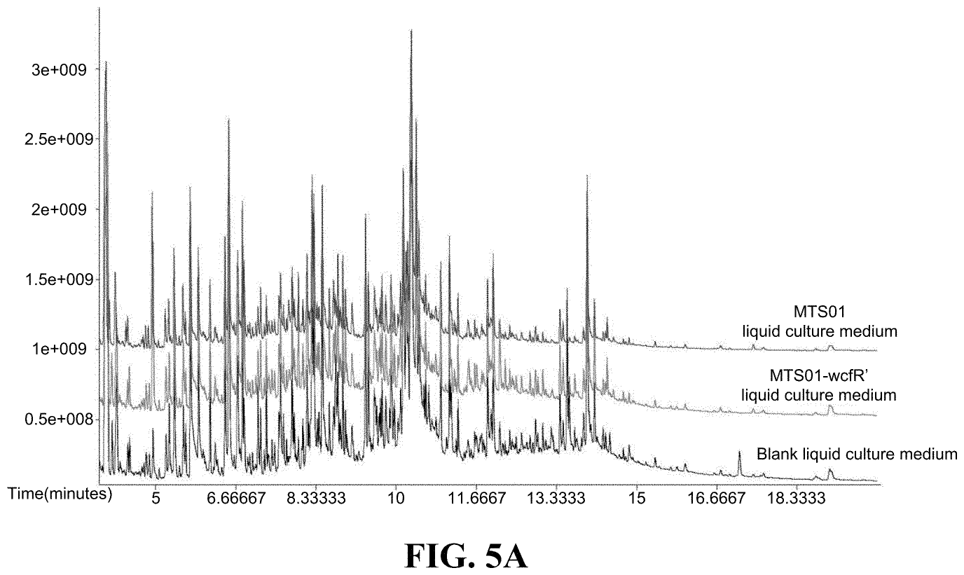

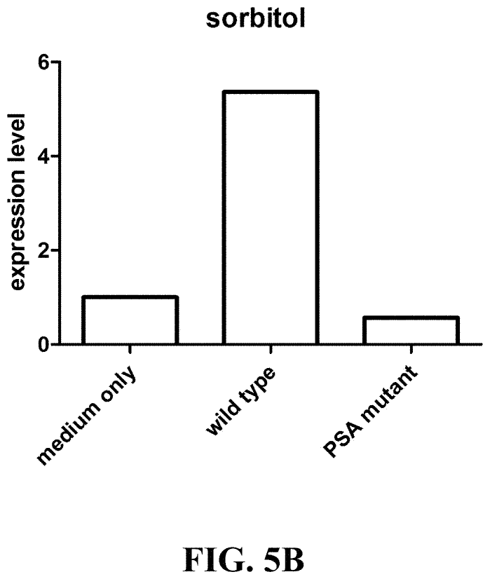

The analysis results of the liquid culture metabolites of the native strain of the Parabacteroides goldsteinii MTS01 and the mutant strain of the Parabacteroides goldsteinii MTS01-wcfR′ with knock out in capsular polysaccharide gene were shown in FIGS. 5A and 5B. As shown in FIG. 5A, there were 2703 analysis signals in total detected in the metabolites of the three culture medium from the native strain of the Parabacteroides goldsteinii MTS01, the mutant strain of the Parabacteroides goldsteinii MTS01-wcfR′, and the blank liquid culture medium control group; wherein, the bottom line was the signals of the blank liquid culture medium control group, the top line was the signals of the native strain of the Parabacteroides goldsteinii MTS01, and the middle line was the mutant strain of the Parabacteroides goldsteinii MTS01-wcfR′. Each signal detected was compared with the mass spectrum signal of the database, and the sorbitol signals detected in the three culture media were significantly different; however, it was not easy to directly separate the differences between each group due to the large number of detected signals in FIG. 5A, so the expression level of sorbitol detected in the three groups were presented in the bar graph as shown in FIG. 5B.

As shown in FIG. 5B, after performing the analysis and the comparison of the differences between the liquid culture metabolites from the three groups, it was found that the content of sorbitol in the metabolites of the native strain of the Parabacteroides goldsteinii MTS01 was significantly higher than that of the mutant strain of the Parabacteroides goldsteinii MTS01-wcfR′; wherein, the content of sorbitol in the blank liquid culture medium control group was used as a comparison standard. The results indicate that the Parabacteroides goldsteinii MTS01 of the present invention can produce sorbitol, and the sorbitol production is regulated by genes involved in regulating capsular polysaccharides.

In summary, the Parabacteroides goldsteinii of the present invention is a multi-functional novel probiotic bacterium. In order to more fully understand the adaptability of the probiotic bacterium to the gastrointestinal tract of individuals, the present invention further analyzes and predicts the genes in the Parabacteroides goldsteinii that are involved in the synthesis of polysaccharides of bacterial capsules and further explore the efficacy or the role in the bacteria of the genes. After the predictive analysis, in the whole genome of the Parabacteroides goldsteinii of the present invention, it is found that the nine-segment CPS regions which may be a sequence fragment of the capsular polysaccharide gene region; wherein, because CPS region A carries a nucleic acid sequence which is similar with the wcfR gene and wcfS gene in the polysaccharide A (PSA) gene region of the Bacteroides fragilis strain NCTC9343, which are mainly responsible for the synthesis of the capsular polysaccharide.

Therefore, this gene region in the Parabacteroides goldsteinii MTS01 is defined as a capsular polysaccharide synthesis gene region, and subsequent studies are carried out. The plasmid containing the gene region A is prepared by conjugation, and the homologous gene recombination method is used to embed the plasmid in the wcfR gene to destroy the coding structure. By polymerase chain reaction, it is confirmed that the plasmid sequence has been successfully embedded in the wcfR gene of the Parabacteroides goldsteinii and caused the destruction of DNA structure. The reverse transcription reaction and polymerase chain reaction have also confirmed that when the DNA structure of the wcfR gene on the Parabacteroides goldsteinii is destroyed by inserting a plasmid in it, the RNA synthesis would indeed be destroyed.

Moreover, a gas chromatograph-time of flight mass spectrometer is used to analyze and compare the differences between the liquid culture metabolites of the native strain of the Parabacteroides goldsteinii MTS01 and the mutant strain of the Parabacteroides goldsteinii MTS01-wcfR′ with knock out in capsular polysaccharide gene. It is found that the content of sorbitol in the metabolites of the native strain of the Parabacteroides goldsteinii MTS01 is significantly higher than that of the mutant strain of the Parabacteroides goldsteinii MTS01-wcfR′, indicating that the Parabacteroides goldsteinii MTS01 of the present invention can produce sorbitol, and the sorbitol production is regulated by genes involved in regulating capsular polysaccharides.

The present invention utilizes the Parabacteroides goldsteinii to produce natural sorbitol to replace the disadvantages caused by industrial manufacturing. At the same time, because the Parabacteroides goldsteinii itself is a multi-functional probiotic bacterium, it can also be combined with its own benefits as probiotics with the function of producing sorbitol to more effectively be used in low-calorie or diet foods, foods for diabetics, preventing dental caries, increasing moisturization, etc. In addition, the present invention can control related sorbitol production genes through regulating the Parabacteroides goldsteinii, and can control the production of sorbitol according to the demand; therefore, the Parabacteroides goldsteinii can be used to prepare sorbitol, and can be used to make a sorbitol-containing composition comprising the a Parabacteroides goldsteinii; wherein, the composition is a food, a drink, a nutritional supplement, a care product, or a medicine, and the composition is in a form of a powder, a granule, a solution, or a gel can be administered to a subject in need by oral administration or the like.

What is claimed is:

1. A method for sorbitol production comprising:
(a) providing a Parabacteroides goldsteinii strain;
(b) cultivating the Parabacteroides goldsteinii strain in a suitable cultivation medium; and
(c) extracting the suitable cultivation medium with an alcohol to form sorbitol.

2. The method according to claim 1, wherein the sorbitol is produced by a gene of capsular polysaccharide of the Parabacteroides goldsteinii strain.

3. The method according to claim 2, wherein the gene of capsular polysaccharide is located in a polysaccharide A gene region of the Parabacteroides goldsteinii strain.

4. The method according to claim 1, wherein the Parabacteroides goldsteinii strain is Parabacteroides goldsteinii DSM32939.

5. A sorbitol-containing composition comprises a Parabacteroides goldsteinii strain.

6. The composition according to claim 5, wherein the sorbitol is produced by the Parabacteroides goldsteinii strain.

7. The composition according to claim 6, wherein the sorbitol is produced by a gene of capsular polysaccharide of the Parabacteroides goldsteinii strain.

8. The composition according to claim 7, wherein the gene of capsular polysaccharide is located in a polysaccharide A gene region of the Parabacteroides goldsteinii strain.

9. The composition according to claim 5, wherein the Parabacteroides goldsteinii strain is Parabacteroides goldsteinii DSM32939.

10. The composition according to claim 5, wherein the sorbitol-containing composition is a food, a drink, a nutritional supplement, a skin care product, or a pharmaceutical product.
